(19) United States
(12) Patent Application Publication
(10) Pub. No.: US 2017/0007315 A1
(43) Pub. Date: Jan. 12, 2017

(54) MANUAL ELECTROCAUTERY DEVICE

(71) Applicant: RAYMED LLC, Wichita, KS (US)

(72) Inventor: Rachel Mary Bender, Wichita, KS (US)

(21) Appl. No.: 14/792,812

(22) Filed: Jul. 7, 2015

(51) Int. Cl. A61B 18/14

(52) U.S. Cl. CPC A61B 18/1402; A61B 2018/00595

(57) ABSTRACT

An electrocautery device includes two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger, and a power unit.

### TECHNICAL FIELD

Example embodiments relate generally to manual electrocautery devices for coagulation and cutting of tissue in a wide variety of surgical applications.

### BACKGROUND

Hysterectomies are the second most common surgical procedure in the U.S, with about 600,000 done annually. The four approaches for surgical removal of a uterus are abdominal, vaginal, laparoscopic, and robotic assisted laparoscopic. Objective evidence supports the total vaginal hysterectomy as the “gold standard” due to its low cost and safety. This approach demands technical skill and experience, which limits its application in many practices. The current instrumentation and approach for a vaginal hysterectomy includes metal clamps and suture ligation. The procedure requires the surgeon to manually identify four ligaments bilaterally to be clamped and sutured. The limitations include poor visualization in a small, cluttered working space. The easy part for the surgeon is to identify the basic anatomy with one's pointer and middle finger, enveloping the appropriate ligament and vasculature. Certain difficulty follows with tracing a Heney clamp along the fingers to grasp all of the selected tissue. The clamp's path is often obstructed with undesired vaginal soft tissue and the surgeon's gloves. This must be done to 4 ligaments bilaterally, in addition to extra vaginal tissue supporting the uterus. This is all done with little to no visualization. Once the clamp is placed, the surgeon must then confidently throw a stitch and tie knots to secure the vessels and tissue while simultaneously removing the clamp—again with very minimal visualization. The total number of knots tied by the surgeon can exceed 300 to secure this small space, which encompasses no more than 3-4 inches circumferentially. Inappropriate timing between removal of the clamp and placement of the stitch leads to devastating consequences including hemorrhage and distorted anatomy. The technical skill required limits its clinical application.

Robotic assist laparoscopic surgery has taken favor among gynecologists due to its technical ease and enhanced visualization and dexterity. Additionally the excitement for a new gadget has created popular demand among patients with disregard to best practice. This fad is not sustainable. A hospital must purchase a $2 million dollar apparatus, surgeons must enroll in hours of training, and an additional $2,200 is spent on each hysterectomy with no improvement in pain control, recovery time, or complications. There is a push among gynecologic surgeons and our pressured healthcare systems to re-embrace the gold standard of care for best practice and cost effectiveness and return to more vaginal surgery.

Therefore there remains a need in the art for a device designed to combine the assets of robotic surgery with the practicality of vaginal surgery to optimize the surgical experience for both patient and physician.

### BRIEF SUMMARY

One or more embodiments of the invention may address one or more of the aforementioned problems. Certain embodiments according to the present invention provide an electrocautery device suitable for a wide variety of applications. In accordance with certain embodiments, the electrocautery device may comprise two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger. The electrocautery device may further comprise a power unit.

### BRIEF DESCRIPTION OF THE DRAWING(S)

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

FIG. 1 illustrates a block diagram of an electrocautery device according to an example embodiment.

FIG. 2 illustrates a top view of two wearable cauterizers worn on a hand according to an example embodiment.

FIG. 3 illustrates a top view of a wearable cauterizer according to an example embodiment.

FIG. 4 illustrates a partial perspective view of an electrocautery device according to an example embodiment.

FIG. 5 a side view of a wearable cauterizer worn on a finger according to an example embodiment.

FIG. 6 illustrates a partial top view of an electrocautery device according to an example embodiment.

### DETAILED DESCRIPTION

Some example embodiments now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

An example embodiment includes an electrocautery device suitable for a wide variety of applications. In accordance with certain embodiments, the the electrocautery device may comprise two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode (e.g., a wire, plate or other conductive surface) disposed between the front bridge structure and the back bridge structure, and an insulator disposed between the cutting electrode and a finger. The electrocautery device may further comprise a power unit.

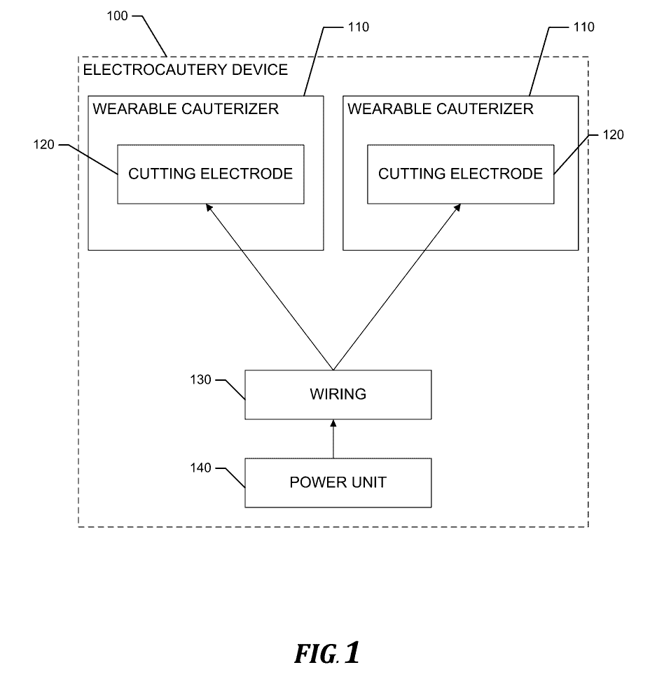

FIG. 1, for example, illustrates a block diagram of an electrocautery device according to an example embodiment. As shown in FIG. 1, the electrocautery device 100 includes two wearable cauterizers 110 having a cutting electrode 120 connected to a power unit 140 via wiring 130. The power unit 140 may be activated by a foot pedal or any other suitable activation means to enable the cutting electrode 120 to surgically coagulate, ligate, and/or cut tissue. The cutting electrode 120 may surgically coagulate, ligate, and/or cut tissue via bipolar cautery, ultrasonographic vibrations, or any other suitable means of coagulating, ligating, and/or cutting tissue.

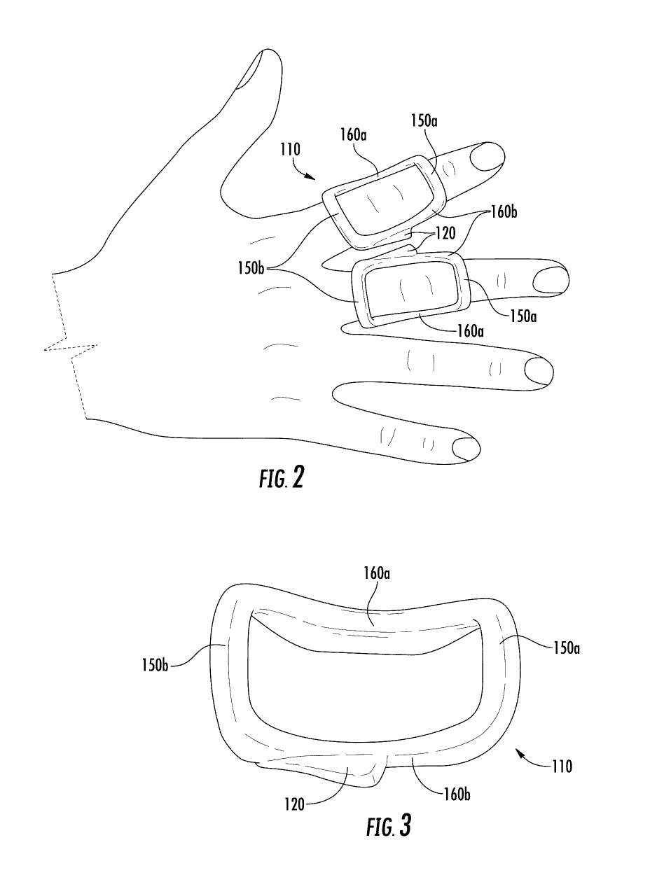

FIG. 2, for example, illustrates a top view of two wearable cauterizers worn on a hand according to an example embodiment. As shown in FIG. 2, each wearable cauterizer includes a cutting electrode 120, a front bridge structure 150a, a back bridge structure 150b, an external connecting member 160a, and an internal connecting member 160b. The wearable cauterizers 110 are worn on the index and middle finger of the hand. Each wearable cauterizer 110 is worn on a finger such that the external connecting member 160a and the internal connecting member 160b extend along opposite sides of the finger.

The wearable cauterizers 110 are positioned on the index and middle fingers respectively such that the cutting electrodes 120 of the wearable cauterizers 110 face each other in the space between the index finger and the middle finger. As such, an electrical current generated by the cutting electrodes 120 is passed between a surgeon's insulated hands. The surgeon's hand may be insulated via at least one of the connecting members 160a, 160b, the surgeon's glove, or a separate insulator. The insulator may selectively line the wiring 130 or may envelop the entire finger depending on the thermal spread generated by the cutting electrodes 120. Additionally, in some embodiments, the wearable cauterizers 100 may include a thermal spread sensor and/or a stopping mechanism to ensure surgeon safety when using the electrocautery device.

FIG. 3, for example, illustrates a top view of a wearable cauterizer according to an example embodiment. As shown in FIG. 3, the wearable cauterizer 110 includes a cutting electrode 120, a front bridge structure 150a, a back bridge structure 150b, an external connecting member 160a, and an internal connecting member 160b. In some embodiments, for example, the front bridge structure 150a and/or the back bridge structure 150b may be a plastic (e.g., lightweight plastic), metal, and/or fabric based on the surgical application for which the electrocautery device 100 will be used and the level of comfort required by the surgeon. If a fabric is used, the fabric may be a neoprene sleeve or any other fabric that has a high thermoinsulative capacity. The external connecting member 160a and/or the internal connecting member 160b may also be plastic, metal, fabric, rubber, or any other suitable material for connecting the front bridge structure 150a and the back bridge structure 150b. In certain embodiments, for instance, the internal connecting member 160b may be the insulator and, as such, is made of an insulating material. In other embodiments, for example, a connecting member may be the cutting electrode, as discussed below in further detail.

FIG. 4, for example, illustrates a partial perspective view of an electrocautery device according to an example embodiment. As shown in FIG. 4, the electrocautery device 100 includes two wearable cauterizers 110 (second wearable cauterizer not shown). The wearable cauterizer 110 includes a cutting electrode 120, a front bridge structure 150a, a back bridge structure 150b, an external connecting member 160a, an internal connecting member 160b, and a passthrough orifice 170. The passthrough orifice 170 extends through an insulator (the internal connecting member 160b in some embodiments) to the cutting electrode 120. As such, the wiring 130 may be built in to the electrocautery device 100, but a user's hand is protected from the wiring 130 due to the passthrough orifice 170 in the insulator. The wearable cauterizer 100 is connected to a power unit (not shown) via wiring 130. The wiring 130 may be secured within a surgeon's glove and gown and also may be grounded to the patient.

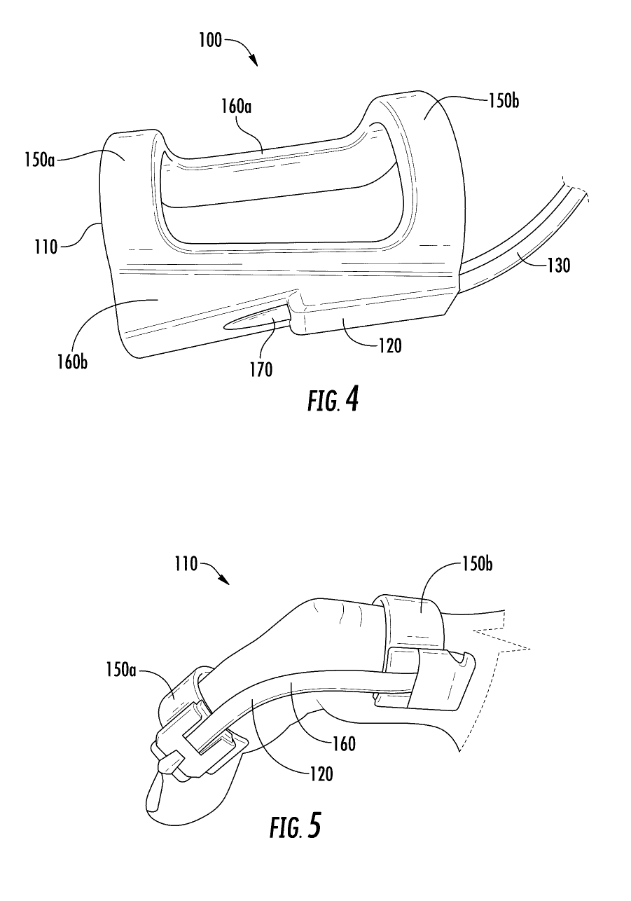

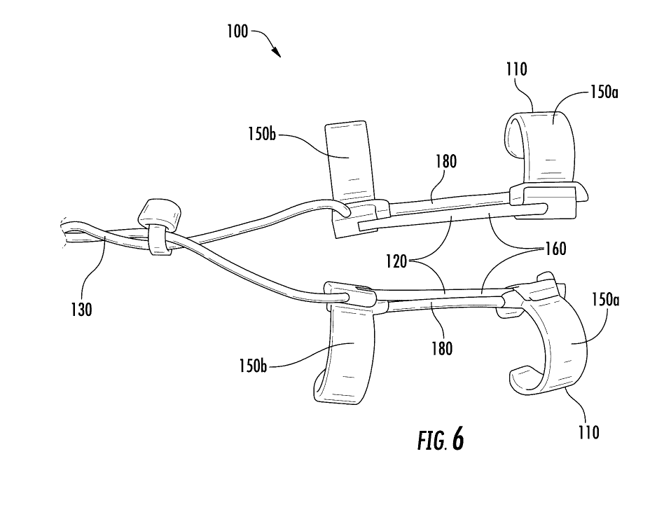

FIG. 5, for example, a side view of a wearable cauterizer worn on a finger according to an example embodiment. As shown in FIG. 5, the wearable cauterizer 110 includes a front bridge structure 150a, a back bridge structure 150b, a cutting electrode 120, and a connecting member 160, such that the cutting electrode 120 is the connecting member 160. The front bridge structure 150a and the back bridge structure 150b extend along both sides of a finger and terminate prior to the palm side of the finger. As such, the palm remains open while using the electrocautery device 100. Additionally, the front bridge structure 150a and the back bridge structure 150b are positioned on the finger so as to allow full joint motion.

FIG. 6, for example, illustrates a partial top view of an electrocautery device according to an example embodiment. As shown in FIG. 6, the electrocautery device 100 includes two wearable cauterizers 110. Each wearable cauterizer 100 includes a front bridge structure 150a, a back bridge structure 150b, a rubber strip 180 (i.e. insulator), a cutting electrode 120, and a connecting member 160, such that the cutting electrode 120 is the connecting member 160. The two wearable cauterizers 110 are connected to a power unit (not shown) via wiring 130.

The rubber strip 180 may be replaced by another insulator including plastic, a glove, or any combination thereof. Alternatively, the insulator may be any other suitable insulating material. In FIG. 6, the rubber strip 180 extends between the front bridge structure 150a and the back bridge structure 150b. Additionally, the connecting member 160 includes the cutting electrode 120 and the rubber strip 180. (i.e. insulator). In some embodiments and as shown in FIG. 6, the cutting electrode 120 extends from the front bridge structure 150a to the back bridge structure 150b and may be entirely disposed between the front bridge structure 150a and the back bridge structure 150b. The cutting electrode 120 may be attached to the front bridge structure 150a and the back bridge structure 150b via a locking mechanism. The locking mechanism may be any suitable means of attaching the cutting electrode 120 to the front and back bridge structures 150a, 150b.

### Exemplary Embodiments

Having described various aspects and embodiments of the invention herein, further specific embodiments of the invention include those set forth in the following paragraphs.

In some example embodiments, an electrocautery device is provided. In general, the electrocautery device, according to certain example embodiments, includes two wearable cauterizers and a power unit. In some cases, each wearable cauterizer comprises a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger. However, in other example embodiments, only one wearable cauterizer may include both the front and rear bridge structures, and the other may include only one such bridge structure with an electrode attached thereto.

In accordance with certain example embodiments, the front bridge structure and the back bridge structure extend along both sides of the finger and terminate prior to a palm side of the finger. In some example embodiments, a first wearable cauterizer is disposed on an index finger and a second wearable cauterizer is disposed on a middle finger. In such embodiments, the cutting electrode of the first wearable cauterizer faces the cutting electrode of the second wearable cauterizer between the index finger and the middle finger.

In accordance with certain example embodiments, a foot pedal activates the power unit to enable the cutting electrode to surgically coagulate and cut tissue. In certain example embodiments, the cutting electrode surgically coagulates and cuts tissue via bipolar cautery. In other example embodiments, the cutting electrode surgically coagulates and cuts tissue via ultrasonographic vibrations.

In accordance with certain example embodiments, the insulator comprises at least one of a rubber strip, plastic, a glove, or any combination thereof. In certain exemplary embodiments, the insulator comprises a rubber strip extending between the front bridge structure and the back bridge structure. In some example embodiments, the at least one connecting member is the insulator.

In accordance with certain example embodiments, the front bridge structure and the back bridge structure comprise plastic. In other example embodiments, the front bridge structure and the back bridge structure comprise metal. In further example embodiments, the front bridge structure and the back bridge structure comprise fabric. In some example embodiments, the at least one connecting member comprises plastic.

In accordance with certain example embodiments, each wearable cauterizer comprises one connecting member. In such embodiments, the connecting member comprises the cutting electrode and a rubber strip disposed between the cutting electrode and the finger. In some example embodiments having one connecting member, the cutting electrode extends from the front bridge structure to the back bridge structure. In certain example embodiments, the cutting electrode is entirely disposed between the front bridge structure and the back bridge structure.

In other example embodiments, each wearable cauterizer comprises two connecting members. In such embodiments, each wearable cauterizer comprises an internal connecting member and an external connecting member, and the internal connecting member and the external connecting member extend along opposite sides of the finger. In some example embodiments, each wearable cauterizer can be modified to further comprise a passthrough orifice extending through the insulator to the cutting electrode.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. An electrocautery device, comprising:
two wearable cauterizers, at least one of the wearable cauterizers comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger; and
a power unit.

2. The electrocautery device according to claim 1, wherein the front bridge structure and the back bridge structure extend along both sides of the finger and terminate prior to a palm side of the finger.

3. The electrocautery device according to claim 1, wherein a first wearable cauterizer is disposed on an index finger and a second wearable cauterizer is disposed on a middle finger, wherein the cutting electrode of the first wearable cauterizer faces the cutting electrode of the second wearable cauterizer between the index finger and the middle finger.

4. The electrocautery device according to claim 1, wherein a foot pedal activates the power unit to enable the cutting electrode to surgically coagulate and cut tissue.

5. The electrocautery device according to claim 1, wherein the cutting electrode surgically coagulates and cuts tissue via bipolar cautery.

6. The electrocautery device according to claim 1, wherein the cutting electrode surgically coagulates and cuts tissue via ultrasonographic vibrations.

7. The electrocautery device according to claim 1, wherein the insulator comprises at least one of a rubber strip, plastic, a glove, or any combination thereof

8. The electrocautery device according to claim 1, wherein the insulator comprises a rubber strip extending between the front bridge structure and the back bridge structure.

9. The electrocautery device according to claim 1, wherein the at least one connecting member is the insulator.

10. The electrocautery device according to claim 1, wherein the front bridge structure and the back bridge structure comprise plastic.

11. The electrocautery device according to claim 1, wherein the front bridge structure and the back bridge structure comprise metal.

12. The electrocautery device according to claim 1, wherein the front bridge structure and the back bridge structure comprise fabric.

13. The electrocautery device according to claim 1, wherein the at least one connecting member comprises plastic.

14. The electrocautery device according to claim 1, wherein each wearable cauterizer comprises one connecting member, and the connecting member comprises the cutting electrode and a rubber strip disposed between the cutting electrode and the finger.

15. The electrocautery device according to claim 14, wherein the cutting electrode extends from the front bridge structure to the back bridge structure.

16. The electrocautery device according to claim 14, wherein the cutting electrode is entirely disposed between the front bridge structure and the back bridge structure.

17. The electrocautery device according to claim 1, wherein each wearable cauterizer comprises an internal connecting member and an external connecting member, and the internal connecting member and the external connecting member extend along opposite sides of the finger.

18. The electrocautery device according to claim 17, wherein each wearable cauterizer further comprises a passthrough orifice extending through the insulator to the cutting electrode.

19. The electrocautery device according to claim 1, wherein both of the wearable cauterizers comprise respective instances of the front bridge structure and back bridge structure.

20. The electrocautery device according to claim 1, wherein only one of the wearable cauterizers includes both the front bridge structure and the back bridge structure, and the other wearable cauterizer includes only a single bridge structure and an electrode extending therefrom.
